(19) United States
(12) Patent Application Publication
(10) Pub. No.: US 2021/0029979 A1
(43) Pub. Date: Feb. 4, 2021

(54) System and Method for the Production of Attractants for Pest Traps

(71) Applicant: Spotta Limited, Cambridge (GB)

(72) Inventors: Robert John Clayton Fryers, Bedford (GB); Gareth David Funk, Cambridge (GB); Neil D'Souza-Mathew, Great Shelford (GB)

(73) Assignee: Spotta Limited, Cambridge (GB)

(21) Appl. No.: 16/961,235

(22) PCT Filed: Jan. 11, 2019
(86) PCT No.: PCT/GB2019/050079

§ 371 Date: Jul. 9, 2020

(30) Foreign Application Priority Data
Jan. 11, 2018 (GB) 1800495.2

(51) Int. Cl. A01K 67/033; A01K 1/01; A01M 1/02

(52) U.S. Cl. CPC A01K 67/033; A01N 63/14; A01M 1/023; A01K 1/01

(57) ABSTRACT

A system/method for producing a substance attractive to one or more species of insect characterised by: housing a population of live insects; providing any combination of nutrients, food or water to the live insects; removing organic matter from the population; and sterilizing the removed organic matter.

### FIELD OF THE INVENTION

The present invention relates to a system i.e. apparatus, and method to produce attractants for use in insect traps to increase the effectiveness of said traps.

### BACKGROUND

The prior art contains an extensive array of insect traps and attraction methods. A common method is to use ultraviolet light to attract insects to a high voltage electrocution grid, or to an adhesive trapping board.

Chemical attractants are also known. For example, U.S. Pat. No. 6,543,181 describes a fruit fly attractant composition comprising: a volatile short chain carboxylic acid, a volatile short chain alcohol, a volatile aryl substituted alcohol, and a nitrogen compound. This document also describes other insect attractants and a portion of the Background Art section of that document is reproduced below verbatim.

“In U.S. Pat. No. 5,490,349, issued Feb. 13, 1996, to Muramatsu, there is disclosed an insect trap which contains a liquid insect attractant which is fully enclosed so that the trap may be shipped without spillage or loss. The trap includes an enclosure which provides a funnel shaped portion terminating in a substantially translucent nipple. The insect trap may be commissioned into service by piercing the nipple thereby creating an inlet into the interior chamber through which insects will be drawn by the liquid attractant. The nipple is formed by a thermal process so that the walls of the inlet are drawn thin creating translucent walls which form a bright spot of light larger than the inlet's opening, thereby making it difficult for trapped insects to find the opening and gain freedom. Except for the terminal portions of the inlet, the enclosure is substantially opaque so that the contents of the insect trap are not visible to the public.

There exists a commercial product that is believed to be an embodiment of the above U.S. Pat. No. 5,490,349, which consists of a small plastic vessel-type trap with a small hole in the top, and named “Natural Catch®0 Plus Fruit Fly Trap”. The liquid attractant used in the trap is believed to be primarily vinegar, which is referred to hereinafter as “Anderson's solution.” In U.S. Pat. No. 5,464,626, issued Nov. 7, 1995, to W. Warren, et al., there is disclosed a method for attracting the insect species Culex nigripalpus, Aedes atianticus, Culex salinarius, Aedes vexans, Culex spp., Simulium spp., Psorofeta ferox, Aedes infirmatus, Drosophila melanogaster, Coccinellidae, Anopheles crucians, Psoroferia columbiae, Culicoides spp. and Aedes spp., using a compound having a dimethyl substituted oxymethyl cyclohexane derivative structure. This attractant finds utility primarily as a bait enhancer for acute toxins and/or trapping devices.

In U.S. Pat. No. 5,008,107, issued Apr. 16, 1991, to C. Warren, et al., there is disclosed a novel attractant composition for use with synanthropic flies (the so-called ‘filth flies’ such as the house fly) which includes indole and skatole, a pheromone, trimethylamine hydrochloride, and a suitable carrier.

In U.S. Pat. No. 6,106,821, issued Aug. 22, 2000, to Baker et al., there is disclosed (house) fly attractant compositions that comprise at least one volatile short chain carboxylic acid, at least one organic sulfide, and at least one nitrogen heterocycle. In a preferred embodiment the composition additionally comprises at least one ammonia-releasing compound. In a particularly preferred example, the composition is prepared by combining, for example, the carboxylic acid, the organic sulfide and the nitrogen heterocycle. The invention also relates to an insect trap comprising a means for retaining flies and an insect attractant composition of this invention. The trap is useful in commercial, residential and livestock facilities.”

As should be relatively clear in light of the number and variety of the attractants available, not all attractants act on a broad spectrum of insects and some are not effective at all. According to the invention defined in U.S. Pat. No. 6,543,181, the attractant is formed of three volatile components and is designed to vaporise at ambient temperature. This leads to evaporation of the liquid and the need to re-fill the trap.

All of the attractants described in the prior art are combinations of specific chemicals, usually synthetic, seeking to produce a chemical combination that attracts the insects by simulating their food, sex pheromones or other attractive substance, usually specific to a target species. However, insects have sophisticated chemoreception capabilities and even well-designed combinations of chemicals are unable to accurately reproduce the full effects of the naturally existing scents they aim to mimic.

Wild insects produce a wide variety of complex chemical mixtures, some of which have the effect of attracting other insects, such as sex or aggregation pheromones.

### SUMMARY OF THE INVENTION

The inventors have recognised problems with known solutions for attracting insects. Such solutions use certain chemicals that are attractive to the target insect but are unable to fully replicate the complex combinations of chemicals that occur naturally and this limits the effectiveness of the known attractants with respect to potency, longevity or efficacy.

This and other aspects will be apparent from the embodiments described in the following. The scope of the present disclosure is not intended to be limited by this summary nor to implementations that necessarily solve any or all of the disadvantages noted.

The present invention comprises a system and method to produce an attractant for use in insect traps where the attractant comprises or consists of biological material produced through the husbandry and/or processing of insects.

Attractants produced from the insects themselves can fully replicate the natural scents that the synthetic attractants in the art seek to mimic; this can enable a much more effective attraction of the target insects to a trap.

It should be noted that in this context an insect trap is not limited to devices that physically confine insects that enter; any device that detects, confines, immobilises or kills insects by any means is considered to be within the scope of the term “trap” used herein.

According to one aspect of the present disclosure there is provided a system for the manufacture of a substance attractive to one or more species of insect. The system comprises a housing containing a live population of insects. The system may further comprise a sub-system to allow nutrients, food and/or water to be provided to the insects. The system may further comprise a sub-system to remove organic matter from the housing. The system may further comprise a sub-system to sterilise the removed material to prevent any ongoing biological activity in the attractant.

The system may further comprise a preparation sub-system to prepare the organic matter into a form appropriate for use as an attractant.

The preparation sub-system may comprise a mechanism to mash or pulps the organic matter.

The system may be configured to produce an attractant from all of or selected parts of insects

The system may be configured to produce an attractant from secretions from the insects

The system may be configured to produce an attractant from sterilised insect eggs

The system may be configured to product an attractant from one or any combination of flies, beetles, bedbugs, moths, bollworms, termites, ants, wasps, hornets, social insects.

The system may be configured to produce an attractant from insects which are the prey of the insects to be attracted.

The system may be configured to produce an attractant from insects which are the same species as the insects to be attracted.

The system may further be configured so that the population of insects is housed in a temperature-controlled environment.

The system may further be configured to sterilise the organic matter by means of heat, cold or radiation to prevent ongoing biological activity in the organic matter.

The system may be further configured to produce an attractant for use in an insect trap or detector.

According to another aspect of the present disclosure there is provided a method for producing a substance attractive to one or more species of insect characterised by: housing a population of live insects; providing any combination of nutrients; food or water to the live insects; removing organic matter from the population and sterilizing the removed organic matter

The method may further be characterised by separating the live insects from the organic matter

The method may further be characterised by heating, cooling or irradiating the organic matter.

The method may further be characterised by producing a substance that is attractive to one or any combination of flies, beetes, bedbugs, moths, weevils, bollworms, termites, ants, wasps, hornets, social insects.

### BRIEF DESCRIPTION OF THE DRAWINGS

FIG. 1 is a diagram of an embodiment for an insect trap with the novel attractant;

FIG. 2 is a system diagram of the preferred embodiment for the system to produce the attractant; and

FIG. 3 is a diagram of an embodiment for a single production cell for attractants suitable for social insects.

### DETAILED DESCRIPTION OF PREFERRED EMBODIMENTS

The present disclosure provides a method, apparatus and system that are practical, inexpensive and allow production of an attractant from the natural biochemistry of insects.

The present disclosure also provides a system/hardware for a trap utilising the novel attractant.

Although the present disclosure is described with respect to presently preferred embodiments relating to the production and use of attractants, it is understood that the features of the present disclosure can be applied to any application requiring the attraction of insects.

The method, apparatus and system enables the production of attractant from the anatomy of insects or their waste. Such attractants may be produced from, but are not limited to, whole insects, casts, eggs, shed skins, excreta or other secretions.

The method, apparatus and system enables to production of a biologically sterile attractant by means of sterilisation. Such sterilisation includes, but is not limited to, heat, cold, ionising radiation and electromagnetic radiation.

The present disclosure generally relates to the production of attractants for use in pest control, and more particularly, but not exclusively, relates to techniques for producing attractants for use in pest traps.

Embodiments will now be described by way of example only.

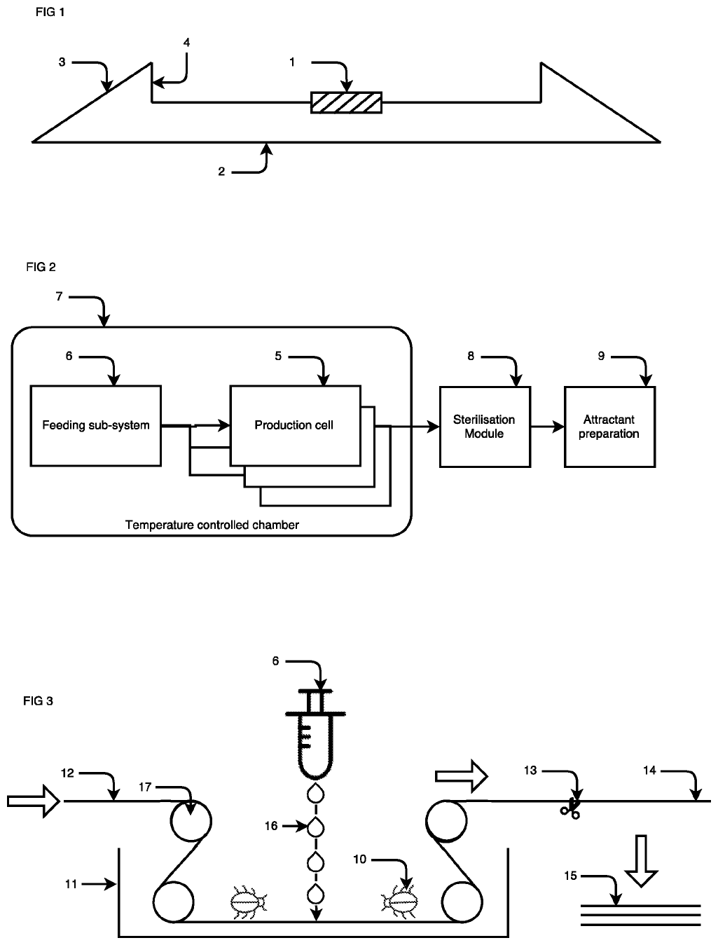

Referring to FIG. 1, an embodiment of a system that advantageously implements the techniques is shown. The system comprises the attractant 1, e.g. produced by means best seen in FIGS. 2 and 3 and described in detail below. The attractant is housed in a housing 2. The housing has features to prevent crawling insects from exiting the trap. The methods of achieving this will be familiar to an expert in the field and in various embodiments may include, but are not limited to: electrical discharge to kill insects, physical features to prevent insect which enter the trap from leaving or adhesives to immobilise insects which enter the trap. FIG. 1 illustrates one possible embodiment where a ramp 3 allow insects to enter the trap, but once within it are unable to climb lip 4 to escape. There are also provided corresponding methods.

Referring to FIG. 2, a block diagram of a preferred embodiment for a system to produce the attractant. A production cell 5 houses a live population of insects. The production cell is best seen in FIG. 3, and is described in detail below. In this system the production cells may be operatively connected to a feeding sub-system which supplies, water, nutrients and other necessary substances to maintain the population of insects in a healthy state. In this embodiment the feeding sub-system and production cells may be maintained at an advantageous temperature for the insect by means of a temperature controlled chamber 7. Periodically, material may be removed from the production cells and sterilised e.g. by means of gamma radiation in a sterilisation module 8. The purpose of this is to kill any live insects or eggs and prevent them from later hatching in the attractant. It will be understood that other embodiments may include alternative means of sterilisation such as heat or microwave radiation. A final stage of attractant preparation 9 may be conducted to process the material into a form suitable for use in a trap. In the preferred embodiment this is through mechanical crushing and/or pulping into a paste. There are also provided corresponding methods.

In other embodiments the production cells may be removable from the temperature controlled chamber and the entirety of their contents sterilised and prepared as attractant. This embodiment is advantageous as an inexpensive system where the entire mass of the insects is effective as an attractant and where the age of the insects is not significant, such as where the insect in the production cell is the prey of the insect to be attracted.

In another embodiment, material is continuously removed from the production cell by means of a conveyer or other continuous system, such as is shown in FIG. 3.

Referring to FIG. 3, an embodiment of a production cell suitable to use to produce an attractant from insect faeces or eggs. Within the production cell a housing 11 retains the insects 10 and prevents them from escaping the cell. In embodiments a strip of material e.g. a continuous strip of material 12, may be passed through the housing e.g. on rollers 17. The rollers may be configured to create a surface(s) at a non-zero angle to horizontal e.g. sufficient to inhibit the insects from climbing up and escaping e.g. greater than 10, 20, 30, 40, 50 or 60 degrees. The material 12 may comprise or consists of a material which the insects find appealing to congregate on. For many social insects, such as ants and bedbugs, filter paper and/or fine cotton are effective for this purpose. In some implementations the feeding sub-system 6 is configured to deliver food for the insects onto the material 12, further encouraging the insects to congregate on the material. The insects on the material will conduct their natural behaviour of feeding and breeding. As a result, the material will become covered in e.g. eggs and/or faeces and/or other material shed from the insects. In implementations the strip of material leaving the production cell may be cut 13 into segments of material e.g. of a suitable size 14 for use in insect traps. Periodically, the produced segments of material 15, may be removed for sterilisation.

A corresponding method may involve providing material e.g. a strip of material at a non-zero angle to the horizontal e.g. as defined by a base of the housing. The method may additionally or alternatively involve passing the (strip of) material though the housing. The method may additionally or alternatively involve using the feeding sub-system to deliver food onto the (strip of) material. The method may further involve harvesting a product of the insects from the (strip of) material; the product may be e.g. eggs, faces, skin/exoskeleton, or other insect product. The collected product, either on/associated with that (strip of) material of after removal from the material, may then be sterilized.

In other embodiments the production cell the system may include the ability to separate mature insects from the production cell for processing into attractant. This may be accomplished by, but is not limited, means of graded sieves to separate larger insects from smaller.

The techniques described herein may also be used with arachnida in particular acari such as mites and ticks.

No doubt many other effective alternatives will occur to the skilled person. It will be understood that the invention is not limited to the described embodiments and encompasses modifications apparent to those skilled in the art lying within the spirit and scope of the claims appended hereto.

1. A system to manufacture a substance attractive to one or more species of insect, comprising:
a housing containing a live population of insects;
a sub-system to allow a combination of nutrients, food, or water to be provided to the live population of insects;
a sub-system to remove organic matter from the housing; and
a sub-system to sterilise the removed organic matter to prevent any ongoing biological activity in the attractant.

2. The system of claim 1, wherein the organic matter removal sub-system is configured to separate live insects from the organic matter.

3. The system of claim 1, comprising a preparation sub-system configured to prepare the organic matter into a form appropriate for use as an attractant.

4. The system of claim 1, wherein the preparation sub-system is configured to slice, mash, or pulp the organic matter.

5. The system of claim 1, wherein the organic matter contains all or selected parts of the insects or their eggs.

6. The system of claim 1, wherein the organic matter consists of secretions from the insects.

7. The system of claim 1, wherein the substance is attractive to one or any combination of flies, beetles, bedbugs, moths, bollworms, termites, ants, wasps, hornets, and social insects.

8. The system of claim 1, wherein the live population of insects contained within the housing is prey of an insect to be attracted.

9. The system of claim 1, wherein the housing is held at a constant temperature.

10. The system of claim 1, wherein the sterilisation sub-system operates by means of applying any combination of heat, cold, or radiation to the organic matter.

11. The system of claim 1, wherein the attractive substance is configured for use in an insect trap or detector.

12. A method for producing a substance attractive to one or more species of insect, the method comprising:
housing a population of live insects;
providing any combination of nutrients, food or water to the population of live insects;
removing organic matter from the population of live insects; and
sterilizing the removed organic matter.

13. The method of claim 12, comprising:
separating the live insects from the organic matter.

14. The method of claim 12, wherein sterilizing the removed organic matter is conducted by heating, cooling, or irradiating the organic matter.

15. The method of claim 12, wherein the substance is attractive to one or any combination of flies, beetles, bedbugs, moths, weevils, bollworms, termites, ants, wasps, hornets, social insects.
